(19) United States
(12) Patent Application Publication
(10) Pub. No.: US 2015/0320676 A1
(43) Pub. Date: Nov. 12, 2015

(54) TOPICAL DRUG FOR TREATING APHTHAE

(71) Applicant: LTS LOHMANN THERAPIE-SYSTEME AG, Andernach (DE)

(72) Inventor: Rosemarie Keikert, Neuwied (DE)

(73) Assignee: LTS Lohmann Therapie-Systeme AG, Andernach (DE)

(21) Appl. No.: 14/652,079

(22) PCT Filed: Dec. 13, 2012
(86) PCT No.: PCT/EP2013/076497

§ 371 Date: Jun. 12, 2015

(30) Foreign Application Priority Data
Dec. 13, 2012 (EP) 12197073.5

(51) Int. Cl. A61K 9/00; A61K 47/36; A61K 47/32; A61K 9/70; A61K 36/48

(52) U.S. Cl. CPC A61K 9/006; A61K 9/7007; A61K 36/48; A61K 47/32; A61K 47/36

(57) ABSTRACT

Topical drugs for treating aphthae, wherein the topical drugs are in the form of a film-shaped dosage form. The topical drugs include a film made of a water-swellable or water-soluble polymer or polymer mixture, and at least one active ingredient selected from the group of tanning agents and essential oils.

The present application claims priority from PCT Patent Application No. PCT/EP2013/076497 filed on Dec. 13, 2013, which claims priority from European Patent Application No. EP 12197073.5 filed on Dec. 13, 2012, the disclosures of which are incorporated herein by reference in their entirety.

### FIELD OF THE INVENTION

The present invention relates to topical medicaments for treating aphthae and in particular topical medicaments provided in a film-shaped form.

It is noted that citation or identification of any document in this application is not an admission that such document is available as prior art to the present invention.

“Aphtha” denotes a painful damage of a mucous membrane bounded by an inflammatory margin. Aphthae are ulcers with a whitish fibrin coating. Small aphthae have a diameter of less than one centimeter and heal mostly within one to two weeks. In rare cases aphthae may be larger than one centimeter, up to 3 cm in diameter. These aphthae denoted as major form may persist for weeks or even months and only heal with scar formation. Aphthae mainly occur on the mucous membrane of the gums, tonsils and tongue. Less common mucous membranes of the genital area are affected by aphthae.

Aphthae are painful. The pain of an aphtha is less correlated with its size, it is rather dependent on the location where the aphtha occurs. In particular during speaking, eating and swallowing the pain becomes noticeable. Therefore, aphthae significantly affect the well-being of those affected. In this respect, a treatment of aphthae is desired, even if they heal by themselves, in order to be free from pain more quickly. For the symptomatic treatment of aphthae analgesics such as lidocaine, polidocanol or benzydamine are offered in the form of sprays, gargle solutions, gels or ointments. Also astringents such as rhubarb root extract, myrrh tincture, silver nitrate, phenolsulfonic acid compounds, the cresol-sulfonic acid condensate policresulen, zinc sulfate or hydrogen peroxide solution are administered. As a prescription agent for the treatment of aphthae the anti-inflammatory triamcinolone acetonide may be mentioned. Alternative anti-inflammatory agents, which are used in the treatment of aphthae are, for example, Australian tea tree oil, melissa extract and chamomile or sage tea.

The unexamined patent publication DE 10 2004 002 001 A1 discloses the use of hyaluronic acid for the treatment of inflammatory diseases. In addition to other forms of administration also occlusive films are mentioned. These occlusive films including hyaluronic acid are applied onto the skin to cover the inflamed area. Aphthae are mentioned as one of the inflammatory diseases which should be treatable with hyaluronic acid.

The document WO 90/01046 A1 describes preparations which are sprayed onto the mucous membrane in order to form a film there.

The document DE 696 13 840 T1 relates to tablets comprising a non-anesthetizing active ingredient core exhibiting a long-term effect, which is provided with an anesthetizing coating. The core may have a hydrophilic surge layer which may comprise a hydrophilic polymer such as Carbopol. In addition to the tablets an active ingredient containing polymer film for the treatment of aphtha is mentioned which absorbs moisture from the buccal mucosa and adheres to the membrane surface. However, it is described as disadvantageous that an irritation of the treatment site when administering an active ingredient leading to irritation is stronger if the administration is implemented by means of such polymer films. This effect is particularly problematic when the treatment site is a painful inflammation, because the pain is increased.

The patent document DE 694 29 964 T2 discloses solid preparations for the treatment of mucosal diseases. These solid preparations are pellets containing a homopolymer or copolymer of acrylic acid, a cellulose ether and gelatine.

In the document US 2007/0104783 A1 the use of orodispersible tablets which are directly adhered to the lesion of a mucous membrane such that their application will certainly be painful for the patient, is mentioned.

The document WO 2003/039465 A2 discloses a patch for the treatment of aphthae wherein a film-shaped medicament is fixed in the mouth by means of a cover patch.

The document US 2007/0248655 A1 describes a patch for administering topically effective drugs in the mouth, wherein exclusively plant polymers are used. The material for producing the patch remains fixed within the mouth and is adapted to be solved slowly in saliva.

The medicaments known from the prior art used for the treatment of aphtha are not optimal in all respects. Thus, ointments, mouthwash, tinctures and pastes have the disadvantage that they only adhere for a short time on the aphtha. Adhesive tablets have the drawback that their application is painful for the patient, because they are hard pellets which exert pressure on the aphtha. Therefore, there is a need for a topical medicament which is adapted to be easily applied, which has a long dwell time on the aphtha and the application of which does not result in even more pain to the patient.

### SUMMARY OF THE INVENTION

Thus, it is an object of the present invention to provide a topical medicament for the treatment of aphtha, which continuously dispenses one or more active ingredients, does not melt within the mouth and ensures that the active ingredient or the active ingredients sufficiently disperse within the oral mucous membrane, and which avoids skin irritations as much as possible during its application.

The object is achieved by a topical medicament in the form of a film-shaped dosage form comprising a water-swellable or water-soluble polymer or polymer mixture and at least one active ingredient suitable for the treatment of aphthae. The film-shaped dosage form according to the invention can easily be applied on an aphtha, may remain there and then release continuously the active agent contained therein, because it dissolves over a longer period, i.e. over a period of more than one hour. Surprisingly, the application of the dosage form according to the invention causes no additional pain for the patient.

In a first aspect the invention relates to a topical medicament for the treatment of aphthae, which is provided as a film-shaped dosage form.

In a second aspect the invention relates to a method for producing the film-shaped dosage form for the treatment of aphthae.

In a third aspect the invention relates to the use of the film-shaped dosage form for the treatment of aphthae.

In a fourth aspect, the invention relates to a method for the treatment of aphthae by means of the film-shaped dosage forms.

### DETAILED DESCRIPTION OF EMBODIMENTS

It is to be understood that the descriptions of the present invention have been simplified to illustrate elements that are relevant for a clear understanding of the present invention, while eliminating, for purposes of clarity, many other elements which are conventional in this art. Those of ordinary skill in the art will recognize that other elements are desirable for implementing the present invention. However, because such elements are well known in the art, and because they do not facilitate a better understanding of the present invention, a discussion of such elements is not provided herein.

The present invention will now be described in detail on the basis of exemplary embodiments.

According to the first aspect of the invention the topical medicament for the treatment of aphthae is provided in the form of a film-shaped dosage form.

The film-shaped dosage form comprises a water-swellable or water-soluble polymer or a water-swellable or water-soluble polymer blend. Furthermore, the film-shaped dosage form comprises at least one active ingredient for the treatment of aphthae, i.e. at least one active ingredient suitable for the treatment of aphthae.

The film-shaped dosage form according to the invention has such an inherent tackiness that a continuous contact with the mucous membrane is guaranteed. The inherent tackiness is provided, because the humidity of the mucous membrane is absorbed by the polymers of the film-shaped dosage form. As a result, they begin to swell. Furthermore, the film-shaped dosage form comprises an expandable polymer film, wherein the at least one active ingredient is present in the polymer or polymer blend in a dissolved or finely dispersed form.

The water-swellable or water-soluble polymer may be a natural polymer, i.e. a polymer which can be found in nature. Natural polymers in the context of the present description also include polymers on the basis of polymers which can be found in nature, which are modified by chemical and/or physical processing(s). According to another embodiment of the film-shaped dosage form the water-swellable or water-soluble dosage form is a fully synthetic polymer. Fully synthetic polymers may be preferred over natural polymers due to their swelling and/or solubility properties if certain embodiments of the film-shaped dosage form are to have specific characteristics.

In embodiments in which the film-shaped dosage form includes a water-swellable or water-soluble polymer blend at least one of the polymers of the polymer blend is a water-swellable or water-soluble polymer. Preferably the at least one water-swellable or water-soluble polymer of the water-swellable or water-soluble polymer blend is a fully synthetic polymer. In another and/or additional embodiment the water-swellable or water-soluble polymer blend can include a plurality of water-swellable or water-soluble polymers, preferably a plurality of fully synthetic water-swellable or water-soluble polymers.

In one embodiment the film-shaped dosage form can include at least one water-swellable or water-soluble polymer selected from the group of polymers consisting of dextran, cellulose derivatives, polyacrylic acid, polyacrylates, polyethylene oxide polymers, polyacrylamides, polyethylene glycol, collagen, alginates, pectins, tragacanth, chitosan, alginic acid, arabinogalactan, galactomannan, agar-agar, agarose, carrageen and natural gums. The cellulose derivatives may be selected from the group consisting of carboxymethylcellulose, ethylcellulose and propylcellulose.

In a further and/or alternative embodiment the film-shaped dosage form may contain at least one water-soluble or at least partially water-soluble polymer selected from the group of polymers consisting of polyvinyl alcohol, cellulose derivatives, starch, starch derivatives, gelatin, polyvinylpyrrolidone, gum arabic, pullulan and acrylates. The cellulose derivatives are preferably selected from the group consisting of hydroxypropyl cellulose, hydroxypropylmethyl cellulose, sodium carboxymethyl cellulose, methyl cellulose, hydroxyethyl cellulose and hydroxypropylethyl cellulose. The starch or starch derivatives can be selected from the group consisting of maltodextrins, amylose, amylopectin, corn starch, potato starch, rice starch, tapioca starch, pea starch, sweet potato starch, barley starch, wheat starch, waxy maize starch and modified starch.

The physical properties of the film-shaped dosage form such as mucoadhesiveness, flexibility, solubility behavior, swelling behavior and the like can be adjusted according to the wishes and needs by combining two or more of the aforementioned polymers.

The physical properties of the film-shaped dosage form may also be adjusted by means of the polymer content. The polymer content of the film-shaped dosage form is at least 5 wt.-%, preferably at least 10 wt.-%, particularly preferably at least 30 wt.-%. The polymer content of the film-shaped dosage form is not more than 90 wt.-%, preferably not more than 70 wt.-%, and particularly preferably not more than 60 wt.-%.

The dosage form according to the invention comprises at least one active ingredient suitable for the treatment of aphthae. The at least one active ingredient is present in the polymer or polymer blend in a dissolved or finely dispersed form.

In a further and/or alternative embodiment the at least one active ingredient may be a tanning agent. A tanning agent means a rather complicated structure of chemical substances, which can be found in many plants. Plants and/or plant parts which include tanning agents and can be used for the production of extracts containing tanning agents may, for example, be selected from the group consisting of gallnuts, witch hazel leaves, walnut leaves, oak bark, rathania root, bloodroot root, blueberry, blackberry, silverweed, strawberry leaves, agrimony, lady's mantle, broadleaf plantain leaves, buckhorn plantain leaves, rose leaves and burnet leaves.

Tanning agents react with the upper layer of the mucous membrane and form a kind of solidified skin proteins (tanning). A tanning agent binds with protein when it comes into contact therewith. The water bound in proteins can be displaced by tanning agents, so that a dehydration is caused. Biologically active proteins can be denatured by tanning agents, such that they are no longer biologically active.

In a further and/or alternative embodiment, the tanning agent may be catechu, i.e. a tanning agent selected from the group of tanning agents of the catechin type. The term catechu actually is the Indian expression for “tree sap”. With respect to the dosage forms described herein catechu means the extracts of dye plants including a high content of condensated tanning agents of the catechin type. As the tanning agent catechu the following catechin types come into consideration:
1. Bengal catechu derived from the red heartwood of Gerber acacia (Acacia catechu), Acacia suma or areca palm;
2. Gambi(e)r catechu derived from leaves and stems of Uncaria gambier bush, which when melted with 1% potassium bicarbonate is called prepared catechu;
3. Mongrove catechu derived from the mangrove Ceriops candolleana;
4. New catechu derived from European softwoods;
5. “Khair” derived from Acacia nilotica, which is used in India for direct browning of silk.

Catechu is usually obtained by boiling the wood or plant parts, such that a resinous extract is obtained which is very tannic. Catechu is soluble in boiling water and typically contains about 2-10% catechin, 25-35% catechin tanning agents, 20-30% quercetin and mucilages and resins. Therefore, catechu means not a single, specific chemical compound, but a composition of various compounds.

In another and/or additional embodiment the at least one active ingredient may be an essential oil. An essential oil means volatile, lipophilic plant compounds with an acceptable inherent smell. Essential oils have an antimicrobial activity. In one embodiment the essential oil may be selected from the group of essential oils included in camomile tincture, sage tincture, melissa tincture or arnica tincture.

In a further and/or alternative embodiment the film-shaped dosage form can include an active ingredient content of at least 0.1 wt.-%, preferably at least 0.5 wt.-%. The film-shaped dosage form includes an active ingredient content of not more than 50 wt.-%, preferably not more than 20 wt.-%.

A single dosage form preferably includes at least 0.5 mg of an active ingredient, preferably at least 1 mg of an active ingredient. A single dosage form includes not more than 20 mg of an active ingredient, preferably not more than 10 mg of an active ingredient.

In a further and/or alternative embodiment the film-shaped dosage form may comprise one or more pharmaceutically acceptable excipients. The type and the amount of excipients also depend on the polymer or polymer blend the dosage form comprises, such as on the at least one active ingredient.

The at least one pharmaceutically acceptable excipient may be selected from the group consisting of plasticizers, stabilizers, carriers, flavorings and fillers.

In a further and/or alternative embodiment the film-shaped dosage form may comprise at least one plasticizer as an excipient. The at least one plasticizer may be selected from the group of plasticizers consisting of glycerin, higher alcohols such as dodecanol, esters of carboxylic acids, in particular esters of carboxylic acids in which the alcohol component is a polyethoxylated alcohol, esters of dicarboxylic acids and triglycerides, in particular medium-chain triglycerides of the caprylic and/or capric acid of coconut oil, propane-1,2-diol. Other polyethylene glycols are mentioned in the pharmacopoeia. The term “higher alcohols” means alcohols which are solid at room temperature, i.e. at a temperature of 18° C. to 22° C. Higher alcohols in the sense of the present invention are preferably physiologically harmless alcohols. The term “medium-chain” in connection with triglycerides denotes the length of the fatty acid residues. Herein, medium-chain triglycerides include fatty acid residues of medium length, i.e. fatty acid residues based on fatty acids including 6 to 12 carbon atoms.

The content of at least one pharmaceutically harmless excipient in the film-shaped dosage form can be more than 1 wt.-% and up to 10 wt.-%, up to 20 wt.-% or up to 30 wt.-%.

In a further and/or alternative embodiment the film-shaped dosage form may have a uniform thickness in a range of 10 μm to 500 μm, preferably in a range of 20 μm to 300 μm.

The individual dosage forms have a surface area in the range between 1 cm² and 10 cm², preferably in the range between 5 cm² and 8 cm².

In a further and/or alternative embodiment the topical medicament may include a releasable protective layer on which the film-shaped dosage form rests. The releasable protective layer may be siliconized paper.

In a further and/or alternative embodiment the film-shaped dosage form may rest directly on the inside of the packaging, i.e. on the surface of a packaging means, which provides a region of the interior of the package, with which the film-shaped dosage form is packed, for the film-shaped dosage form.

In a further and/or alternative embodiment the releasable protective layer with which the film-shaped dosage form is in contact, can be made from the same materials that are used for the production of process liners, inasfar the latter have been made releasable, for example by siliconisation. A process liner is a film, which is used in the manufacturing process of planar medicament forms, but is not present in the end product because it has been removed according to the process. The releasable protective layer may be a layer of polytetrafluoroethylene, treated paper, cellophane, polyvinyl chloride or the like.

In a further and/or alternative embodiment the releasable protective layer comprises a surface area which is larger than the surface area of the film-shaped dosage form having a size and format appropriate for therapy. Preferably, the larger surface area of the releasable protective layer is configured such that it protrudes substantially at one end beyond the surface of the film-shaped dosage form. This protruding region of the releasable protective layer enables the protective layer to be pulled off easier from the film-shaped dosage form, in particular during the application of the film-shaped dosage form.

In a further and/or alternative embodiment the dosage form is packed individually dosed in a sealed edge bag. According to another embodiment several dosage forms are provided in a common package.

As a packaging material for the film-shaped dosage forms preferably sealable composite packaging materials are used. Preferred sealing media which comprise the sealable composite packaging material on those of their sides, which forms the interior of the later packaging, are polyacrylonitrile and polyethylene terephthalate, because they absorb no or only a very small amount of essential oil during the storage of the packed film-shaped dosage forms.

According to the second aspect the invention provides a method for producing the film-shaped dosage forms described above.

In one embodiment the film-shaped dosage form according to the invention can be prepared by homogeneously dispersing the at least one active ingredient in a solution or suspension of the polymer or the polymer blend in a solvent or suspension agent.

In a further and/or alternative embodiment the at least one active ingredient together with the at least one pharmaceutically acceptable excipient can be homogeneously dispersed in a solution or suspension of the polymer or the polymer blend in a solvent or suspension agent.

The resulting mixture of a polymer or polymer blend, at least one active ingredient and optionally at least one pharmaceutically acceptable excipient can be coated onto a carrier material. Then the solvent or suspension agent can be removed from the coated mixture, such that a polymer film is obtained, from which the film-shaped dosage forms can be separated.

According to the third aspect the invention includes the use of film-shaped dosage forms described above for the treatment of aphthae. To this end, one of the film-shaped dosage forms can be applied onto the aphtha to be treated, such that the aphtha is covered by the film-shaped dosage form. Due to its mucoadhesive properties the film-shaped dosage form remains in contact with the mucous membrane and releases the at least one active ingredient it contains to the affected mucous membrane as well as to the vicinity of the aphtha. The film-shaped dosage form dissolves over a longer period of time.

According to the fourth aspect the invention also encompasses methods for the treatment of aphthae, in particular for the treatment of aphthae in the oral cavity, in which a film-shaped dosage form consisting of a film of a swellable or water-soluble polymer or polymer blend and at least one active ingredient selected from the group consisting of tanning agents and essential oils, is applied onto the aphtha to be treated such that the film-shaped dosage form covers the aphtha.

The present invention is illustrated below with respect to exemplary embodiments which only serve for explanation and are not intended to limit the present invention. The present invention is exclusively defined by the claims.

### Example 1

40 g catechu (powdered), 10 g cinnamon powder and 150 g polyvinylpyrrolidone K 90 were dissolved in 300 ml of 45% ethanol with stirring. After complete dissolution of the solids a viscous liquid was obtained which was applied onto siliconized paper by means of a squeegee roller. By mild drying the majority of the water and the ethanol was removed from the viscous liquid, such that a film was obtained. Subsequently, by use of a hollow punch circular structures with an area of about 1 cm² were punched out and placed with their film side onto the sealing medium of a composite packaging material. Then the siliconized paper was removed and another layer of the composite packaging material was placed onto the circular film such that the sealing surfaces of the two composite packaging material layers were facing each other. By means of a sealing mask the composite packaging material layers were welded to obtain a sealed edge bag which contained the film containing the active ingredient.

### Example 2

A film-shaped dosage form according to Example 1 was prepared, wherein the film included the following composition:
1.0 mg catechu tincture
6.5 mg Carbopol
2.5 mg starch.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

It is further noted that the invention does not intend to encompass within the scope of the invention any previously disclosed product, process of making the product or method of using the product, which meets the written description and enablement requirements of the USPTO (35 U.S.C. 112), such that applicant(s) reserve the right to disclaim, and hereby disclose a disclaimer of, any previously described product, method of making the product, or process of using the product.

While this invention has been described in conjunction with the specific embodiments outlined above, it is evident that many alternatives, modifications, and variations will be apparent to those skilled in the art. Accordingly, the preferred embodiments of the invention as set forth above are intended to be illustrative, not limiting. Various changes may be made without departing from the spirit and scope of the inventions as defined in the following claims.

1. Topical medicament for the treatment of aphthae, including a film-shaped dosage form comprising a film of a water-swellable or water-soluble polymer or a water-swellable or water-soluble polymer blend and at least one active ingredient for the treatment of aphthae.

2. Topical medicament according to claim 1, characterized in that the water-swellable or water-soluble polymer or at least one water-swellable or water-soluble polymer of the water-swellable or water-soluble polymer blend is a fully synthetic polymer.

3. Topical medicament according to claim 1, characterized in that the film-shaped dosage form comprises at least one water-swellable or water-soluble polymer selected from the group consisting of dextran, cellulose derivatives, polyacrylic acid, polyacrylates, polyethylene oxide polymers, polyacrylamides, polyethylene glycol, collagen, alginates, pectins, tragacanth, chitosan, alginic acid, arabinogalactan, galactomannan, agar-agar, agarose, carrageenan and natural gums.

4. Topical medicament according to claim 1 or 3, characterized in that the film-shaped dosage form comprises at least one water-soluble or at least partially water-soluble polymer selected from the group consisting of polyvinyl alcohol, cellulose derivatives, starch, starch derivatives, gelatin, polyvinylpyrrolidone, gum arabic, pullulan and acrylates.

5. Topical medicament according to any one of the preceding claims, characterized in that the polymer content in the film-shaped dosage form is at least 5 wt.-%, preferably at least 10 wt.-%, particularly preferably at least 30 wt.-%, and not more than 90 wt.-%, preferably not more than 70 wt.-%, and particularly preferably not more than 60 wt.-%.

6. Topical medicament according to any one of the preceding claims, characterized in that the at least one active ingredient is selected from the group consisting of tanning agents and essential oils.

7. Topical medicament according to claim 6, characterized in that the tanning agent is selected from the group of catechin tanning agents.

8. Topical medicament according to claim 6 or 7, characterized in that the tanning agent is catechu.

9. Topical medicament according to claim 6, characterized in that the essential oil is selected from the group of essential oils which are included in camomile tincture, sage tincture, melissa tincture or arnica tincture.

10. Topical medicament according to any one of the preceding claims, characterized in that the film-shaped dosage form has an active ingredient content of at least 0.1 wt.-%, preferably at least 0.5 wt.-%, and not more than 50 wt.-%, preferably not more than 20 wt.-%.

11. Topical medicament according to any one of the preceding claims, characterized in that the film-shaped dosage form comprises at least one pharmaceutically acceptable excipient which is preferably selected from the group consisting of plasticizers, stabilizers, carriers, flavorings and fillers.

12. Topical medicament according to claim 11, characterized in that the plasticizer is selected from the group of plasticizers consisting of glycerol, alcohols which are solid at room temperature, such as dodecanol, esters of carboxylic acids, in particular esters of carboxylic acids in which the alcohol component is a polyethoxylated alcohol, esters of dicarboxylic acids and triglycerides, in particular medium-chain triglycerides of the carbryl and/or capric acid of coconut oil, propane-1,2-diol and other polyethylene glycols.

13. Topical medicament according to claim 11 or 12, characterized in that the content of the at least one pharmaceutically acceptable excipient is more than 1 wt.-% and up to 30 wt.-%.

14. Topical medicament according to any one of the preceding claims, characterized in that it comprises a releasable protective layer on which the film-shaped dosage form rests.

15. Method for producing a topical medicament according to any one of claims 1 to 14, characterized in that in the method the at least one active ingredient and optionally the at least one excipient are dispersed homogeneously in a solution or suspension of the polymer or the polymer blend in a solvent or suspension agent, the resulting mixture is coated onto a carrier material, subsequently the solvent or suspension agent is removed such that a polymer film is obtained, and the dosage forms are separated therefrom.

16. Use of a topical medicament according to any one of the preceding claims for the treatment of aphthae.
